(19) United States
(12) Patent Application Publication
(10) Pub. No.: US 2021/0346312 A1
(43) Pub. Date: Nov. 11, 2021

(54) Flat Product for Microbiological Sanitation of the Urogenital Tract

(71) Applicant: Tecuro Medical GmbH, Luzern (CH)

(72) Inventors: Corinna Hengsberger, Schopfheim (DE); Otto von Stetten, Aachen (DE); Gerd Ries, Duisburg (DE)

(21) Appl. No.: 17/273,141

(22) PCT Filed: Sep. 2, 2019
(86) PCT No.: PCT/EP2019/073317

§ 371 Date: Mar. 3, 2021

(30) Foreign Application Priority Data
Sep. 4, 2018 (EP) PCT/EP2018/073776

(51) Int. Cl. A61K 9/70; A61K 31/194; A61K 31/07; A61K 31/375; A61K 31/355; A61K 31/155; A61K 33/18; A61K 33/34; A61K 33/30; A61K 33/26; A61K 33/38; A61K 33/24; A61P 31/04; A61L 15/46; A61L 15/28

(52) U.S. Cl. CPC A61K 9/7084; A61L 2300/404; A61K 31/07; A61K 31/375; A61K 31/355; A61K 31/155; A61K 33/18; A61K 33/34; A61K 33/30; A61K 33/26; A61K 33/38; A61K 33/24; A61P 31/04; A61L 15/46; A61L 15/28; A61K 31/194

(57) ABSTRACT

A planar product for treatment of the urogenital tract having a proximal face and a distal face including at least one textile layer (4). The proximal face being coated with an antimicrobially and/or antiseptically effective agent. The planar product has a vitality of >80% after 18 h of incubation time in a Cytotoxicity Test described herein and an efficacy of <80% in a PI-MIA Test described herein.

### CROSS-REFERENCE TO RELATED APPLICATIONS

This application is the United States national phase of International Application No. PCT/EP2019/073317 filed Sep. 2, 2019, and claims priority to International Application No. PCT/EP2018/073776 filed Sep.. 4, 2018, the disclosures of each of which are hereby incorporated by reference in their entireties.

### BACKGROUND OF THE INVENTION

### Field of the Invention

The present invention relates to the field of hygiene technology in the urogenital area. More particularly, the invention relates to a planar product for treatment of the urogenital tract and for use in the therapy or the prophylaxis of, for example, urinary tract inflammations and other infectious diseases. Furthermore, the present invention relates to a method of production of a planar product for treatment of the urogenital tract.

### Related art

Kidney and urinary tract inflammations are common infectious diseases, with women being affected substantially more frequently than men because of anatomical proportions. About 10% of women in the USA and Europe suffer from recurrent infections with three or more episodes per year. The cause is mainly E. coli bacteria, but also Klebsiellae, Proteus or Staphylococci, which originate from one's own intestine. They migrate from the anus, across the skin, into the urethra and ultimately into the bladder and, from there, into the renal pelvis. Even after an inflammation that has been overcome, niches comprising surviving bacteria can form at the entry point of the urethra, and these can lead to a relapse.

Therapy is often carried out by using systemically applied antibiotics, which, however, become ineffective when used frequently, this being necessary, owing to the bacterial resistances that form, and can have undesired adverse effects. Phytotherapy, which is popular at present and mainly based on lingonberries or cranberry, has shown variable results in studies and validation thereof has so far not been possible. More recent approaches describe methods to prevent the bacteria that are involved from migrating or to eliminate them before they can reach the urethra and hence the bladder or the kidneys.

US patent application US 2002/0115976 proposes a sanitary pad which covers the anal area, the perineum and the genital area of women. Provided in the central perineal area is a mechanical obstacle which runs transversely on the pad. Said obstacle can comprise antimicrobial substances, so that the bacteria cannot supposedly migrate from the anus to the genital area. It is expressly required that the antimicrobial substances do not reach the genital area. However, microorganisms are not stopped by a mechanical obstacle, even if it contains antimicrobial substances, since microorganisms will look for all possible routes to their destination.

WO 2015/154746 proposes a cover for the urogenital area, at least in the region of the vulva, the vagina and the urethra, which is substantially completely prepared with an antiseptic substance to prevent the migration of bacteria into the urogenital tract.

### SUMMARY OF THE INVENTION

The use of antimicrobially and antiseptically effective agents in covers for the urogenital area means that the sensitive mucosas of the urogenital tract come into close contact with these substances. Since such covers are, from experience, used and worn for a relatively long time, a significant problem of such covers are skin irritations, abrasions and intolerances right up to the development of allergies. For a long time, the focus of research has been on searching for a possible substitute for antibiotic agents in order to prevent resistances from forming. Particular attention has been given here to identifying substances which can control the relevant bacteria with identical or at least approximately identical efficacy. However, at the same time, the skin compatibility of such substitute substances has not been taken into account. Known antiseptics are frequently aggressive toward the skin and especially toward the mucosal cells. Although sanitary pads can be provided with moisturizers in order to avoid an increased skin rubbing by the textile layers, they have no effect on the aggressively acting antiseptics. It is therefore an object of the invention to improve the prior art in the area of hygiene technology in the urogenital area. In advantageous embodiments, the present invention can provide a device for treatment of the urogenital tract that has an improved skin compatibility and preferably a sufficient efficacy against bacteria. In further advantageous embodiments, infections of the urogenital tract can be prevented and/or treated in a reliable manner

It is a further object of the invention to improve the prior art in the area of methods of production of devices in hygiene technology. According to advantageous embodiments, what is provided is a cost-effective and/or efficient method of production for a device for treatment of the urogenital tract.

At least one of these objects is achieved in a general manner by the subject matter of the independent claims.

Further advantageous embodiments are apparent in each case from the dependent claims and from the disclosure as a whole.

### BRIEF DESCRIPTION OF THE DRAWINGS

Aspects of the invention will be more particularly elucidated on the basis of the exemplary embodiments shown in the following figures and the associated description. In the Figures:

FIG. 1 shows a schematic view of a planar product according to the invention for treatment of the urogenital tract as per one embodiment of the invention;

FIG. 2 shows a schematic view of a planar product according to the invention for treatment of the urogenital tract as per a further embodiment of the invention;

FIG. 3 shows a schematic view of a planar product according to the invention for treatment of the urogenital tract as per some further embodiments of the invention;

FIG. 4 shows a schematic view of a planar product according to the invention for treatment of the urogenital tract as per some further embodiments of the invention; and

FIG. 5 shows a reference scale for the determination of PI-MA values.

### DETAILED DESCRIPTION

In a first aspect, the invention provides a planar product for treatment of the urogenital tract having a proximal face and a distal face. The planar product includes at least one textile layer. The proximal face forms, in the operative state, the face of the planar product that is facing the skin, and the distal face forms the face of the planar product that is facing away from the skin in the operative state. The proximal face is coated with an antimicrobially and/or antiseptically effective agent, the planar product having a vitality of >80% after 18 hours (h) of incubation time in the cytotoxicity test described below. Furthermore, the planar product has an efficacy of >80% in the PI-MIA test described below.

In one embodiment of the invention, the proximal face of the planar product for treatment of the urogenital tract comprises a top stitching in the center in the longitudinal direction. In the context of the invention, “in the longitudinal direction” means that it runs in the direction of the labia, and not transversely thereto, in the operative state. In embodiments with multiple textile layers, the top stitching can also be configured to join at least two textile layers. In general, a top stitching facilitates bending of the planar product, enlarges the contact area in relation to the skin and prevents slipping of the planar product according to the invention during wearing. Typically, the length of the stitching is substantially ⅔ or more of the length of the planar product.

In further embodiments, the distal face is hydrophobized. To this end, it can either be coated with a hydrophobic material such as, for example, fluorocarbon or consist of such a material. Such embodiments make it possible to prevent possible leakages and moreover ensure that the antimicrobially and/or antiseptically effective agent does not escape from the planar product and that it does not evaporate outwardly or is not passed to clothing in the operative state.

In a further embodiment, at least one textile layer includes a skin-compatible textile, for example of skin-compatible woven fabrics, knitted fabrics or nonwovens. In embodiments with multiple textile layers, they can include different materials, and in this case at least the proximal face typically includes a skin-compatible material. Skin-compatible materials are preferably woven fabrics, knitted fabrics and nonwovens, for example cotton, polypropylene, polyester, polyethylene, polyamide, nylon, etc. The surface of the textile layers, especially of the proximal face, can optionally be structured. Owing to the use of such skin-compatible materials, it is preferably possible to increase wear comfort and to prevent skin irritations. In a typical embodiment, the proximal face is coated with bioadhesive and/or mucoadhesive agents in stripes or in a punctiform manner for better fixation on the skin. Such agents serve to place the planar product tightly on the skin and they thus improve efficacy against bacteria. In addition, the improved adhesion of the planar product reduces rubbing, and this increases wear comfort. Bioadhesive and/or mucoadhesive agents that can be used are, for example, carbomers, poloxamers, polyacrylates and derivatives, chitosans and chitosan derivatives, alginate polyethylene glycol acrylates, thiomers (e.g., polycarbophil-cysteine, etc.), lectins, and also mixtures thereof.

In a preferred embodiment, the antimicrobially and/or antiseptically effective agent includes surfactants, quaternary ammonium compounds, biguanides, organic acids or salts thereof, metal ions such as copper, zinc, iron, silver, ruthenium, rhodium, iodine compounds, aldehydes and mixtures thereof.

In some embodiments, the antimicrobially and/or antiseptically effective agent includes tartaric acid or a salt thereof in a concentration of 1 to 5 g/L, preferably 2 to 4 g/L. More particularly, the tartaric acid can be present in a maximum concentration of 3 g/L, for example 1 to 3 g/L. It has become apparent that tartaric acid in this concentration range has an as advantageous for effect on cell vitality according to the cytotoxicity test described here.

Typically, the antimicrobially and/or antiseptically effective agent includes at least one vitamin, especially vitamin A, B, C, E, at least one metal ion of group eight, eleven or twelve of the IUPAC periodic table, for example iron, copper or zinc, at least one aliphatic or aromatic organic acid or a derivative thereof and at least one surfactant. Optionally, the antimicrobially and/or antiseptically effective agent additionally includes bases and/or buffer systems for pH adjustment. A planar product comprising such an agent satisfies both the requirements of skin compatibility and of efficacy in a particularly satisfactory manner What have been found to be particularly skin-compatible are embodiments which additionally contain bases and/or buffer systems. Surprisingly, the lower acidity of the agent does not lead to a significant decline in antibacterial, antimicrobial or antiseptic efficacy.

The vitamins can be used in their common or ionic forms. Particular preference is given to using vitamin C, riboflavin and/or niacin. In this connection, the concentration of the vitamins can be between 0.5 and 1.5 g/L.

What can serve as a source of metal ions are common salts of the respective metal ions, for example halides, sulfates, nitrates, sulfites or phosphates. Preferred metal ions are copper, iron and zinc. The concentrations vary between 0.5 and 1.5 g/L, preferably 0.75 and 1.25 g/L. These concentrations have been found to be particularly advantageous because they are high enough to ensure a sufficient efficacy against bacteria, but at the same time also do not have an adverse effect on skin compatibility.

Derivatives of organic acids, which can likewise be used as organic acids, can especially include the deprotonated forms thereof. They can be obtained either from the respective salts, for example ammonium, sodium, potassium, magnesium, calcium, lithium, etc., or by deprotonation of the acids with the optionally present bases. Furthermore, such derivatives include ester or amide compounds. Here, typical examples are formic acid, acetic acid, bromoacetic acid, glycolic acid, propionic acid, glyoxylic acid, lactic acid, citric acid, tartaric acid, malonic acid, maleic acid, fumaric acid, pyrrolidone carboxylic acid, sorbic acid, undecylenic acid, undecynoic acid, benzoic acid, hydroxybenzoic acid, salicylic acid, dehydroacetic acid, 4-hydroxybenzoate, dimethyl carbonate, chloroacetamide, 2-chloro-N-(hydroxymethyl)acetamide, salicylanilide. Preference is given to using tartaric acid and citric acid. Particular preference is given to using tartaric acid and citric acid in concentrations of 2 to 6.5 g/L. The surfactants are selected from the group of anionic, nonionic, amphoteric or cationic surfactants. These are, for example, alkyl ether sulfates, alkyl and/or aryl sulfonates, alkyl sulfates, olefin sulfonates, amphosurfactants, betains, alkylamidoalkylamines, alkyl-substituted amino acids and/or imino acids, acylated amino acids, sugar surfactants, quaternary ammonium compounds, etc. The surfactants are used either individually or in suitable mixtures with one another. Preference is given to using anionic surfactants, and particular preference is given to using lauryl sulfates, especially sodium lauryl sulfate. The typical amounts vary between 1.5 and 10 g/L, preferably 2.0 and 8 g/L, particularly preferably 2.5 and 6.5 g/L. Typical buffer systems are, for example, phosphate, acetate, citrate, tris(hydroxymethyl)aminomethane (Tris), succinate, carbonate, borate, oxalate, glycine or tartrate buffer.

In a further embodiment, the antimicrobially and/or antiseptically effective agent includes at least one gel former. Such gel formers are particularly advantageous because it has become apparent that, surprisingly, the release of the effective agent can be controlled via the concentration thereof or via the resulting layer thickness, with the result that a sufficient amount of the effective agent is available for a sufficiently long time without the planar product having to be overloaded with the agent before use. As a result, the duration of efficacy can be significantly improved.

In preferred embodiments, the concentration of the gel former in the antimicrobially and/or antiseptically effective agent is 2.5 to 45 g/L, preferably 5 to 35 g/L, particularly preferably 7.5 g/L to 25 g/L. Typically, the at least one gel former includes a polysaccharide, preferably agarose, carrageenan, pectin, xanthan gum, alginic acid and alginates, silicon dioxide, polyvinylpyrrolidones, polyvinyl alcohols, polymethacrylates, preferably poly(hydroxy methacrylates), poly(N-isopropylacrylamides), polyacrylic acids or mixtures thereof. Here, polysaccharides are particularly preferred gel formers which particularly advantageously have an effect on the release of the agent. Furthermore, it has become apparent that natural gel formers have the advantageous effect of yet further reducing the risk of allergic reactions.

In a preferred embodiment, the antimicrobially and/or antiseptically effective agent contains ascorbic acid as vitamin, copper as metal ions, citric and/or tartaric acid or a salt thereof as organic acid, lauryl sulfates, especially sodium lauryl sulfate, as surfactant and optionally agarose or carrageenan as gel former.

In some preferred embodiments, the antimicrobially and/or antiseptically effective agent contains ascorbic acid as vitamin, copper, iron or zinc as metal ions, citric and/or tartaric acid or a salt thereof as organic acid, lauryl sulfates, especially sodium lauryl sulfate, as surfactant and optionally agarose or carrageenan as gel former. Preferably, the concentration of the tartaric acid in the antimicrobially and/or antiseptically effective agent is 1 to 5 g/L, preferably 2 to 4 g/L.

In a further embodiment, the concentration of the at least one vitamin is 0.5 to 1.5 g/L; and /or

that of the at least one aliphatic or aromatic acid is 2 to 6.5 g/L; and/or

that of the metal ions is 0.5 to 1.5 g/L; and/or

that of the surfactants is 1.5 to 10 g/L, preferably 2 to 8 g/L, particularly preferably 2.5 to 6.6 g/L; and/or

that of the at least one gel former is 2.5 to 45 g/L, preferably 5 to 35 g/L, particularly preferably 7.5 g/L to 25 g/L.

In preferred embodiments, the concentration of the at least one vitamin is 0.5 to 1.5 g/L; and

that of the at least one aliphatic or aromatic acid is 2 to 6.5 g/L; and

that of the metal ions is 0.5 to 1.5 g/L; and

that of the surfactants is 1.5 to 10 g/L, preferably 2 to 8 g/L, particularly preferably 2.5 to 6.6 g/L.

In further embodiments, the concentration of the at least one vitamin is 0.5 to 1.5 g/L; and

that of the at least one aliphatic or aromatic acid is 2 to 6.5 g/L; and

that of the metal ions is 0.5 to 1.5 g/L; and

that of the surfactants is 1.5 to 10 g/L, preferably 2 to 8 g/L, particularly preferably 2.5 to 6.6 g/L; and

that of the at least one gel former is 2.5 to 45 g/L, preferably 5 to 35 g/L, particularly preferably 7.5 g/L to 25 g/L.

In preferred embodiments, the antimicrobially and/or antiseptically effective agent contains ascorbic acid as vitamin in a concentration of 0.5 to 1.5 g/L; and at least one aliphatic or aromatic acid or a salt thereof in a concentration of 2 to 6.5 g/L; and metal ions in a concentration of 0.5 to 1.5 g/L; and surfactants in a concentration of 1.5 to 10 g/L, preferably 2 to 8 g/L, particularly preferably 2.5 to 6.6 g/L.

In further embodiments, the antimicrobially and/or antiseptically effective agent contains ascorbic acid as vitamin in a concentration of 0.5 to 1.5 g/L; and at least one aliphatic or aromatic acid or a salt thereof in a concentration of 2 to 6.5 g/L; copper, iron and/or zinc as metal ions in a concentration of 0.5 to 1.5 g/L; and surfactants in a concentration of 1.5 to 10 g/L, preferably 2 to 8 g/L, particularly preferably 2.5 to 6.6 g/L.

In a further embodiment, the antimicrobially and/or antiseptically effective agent contains ascorbic acid as vitamin in a concentration of 0.5 to 1.5 g/L, copper, iron and/or zinc as metal ions in a concentration of 0.5 to 1.5 g/L, citric and/or tartaric acid as organic acid in a concentration of 2 to 6.5 g/L, especially 2 to 4 g/L, lauryl sulfates, especially sodium lauryl sulfate, as surfactant in a concentration of 2 to 8 g/L, preferably 2.5 to 6.6 g/L. In addition, the antimicrobially and/or antiseptically effective agent can optionally contain agarose or carrageenan as gel former in a concentration of gel former of 2.5 to 45 g/L, preferably 5 to 35 g/L, particularly preferably 7.5 g/L to 25 g/L. It has become apparent that such embodiments have both a very high efficacy and, at the same time, high skin compatibility.

In some embodiments, the proximal face of the planar product according to the invention has an area of 20 to 25 cm², preferably 23 cm². Typically, between 0.3 and 1.8 mL of the antimicrobially and/or antiseptically effective agent is applied to such an area in further embodiments. This corresponds to a doping of 0.015 to 0.078 mL/cm². Such a value is also advantageous in the case of other areal values of the proximal face of a planar product according to the invention and hence not restricted to an area of 20 to 25 cm². A doping of 0.015 to 0.078 mL/cm² is, firstly, low enough for the planar product not to be permeated by the liquid and hence not to reduce the wear comfort and, secondly, great enough for a sufficient and effective amount to be available for controlling bacteria.

In a preferred embodiment, the antimicrobially and/or antiseptically effective agent has a pH between 1.8 and 5.2, preferably 2.0 to 4.2, especially 2.3 to 3.1. As a result, skin compatibility can be additionally increased and irritations can be avoided. Surprisingly, this effect becomes particularly noticeable at a pH of 2.3 to 3.1, this being significantly below the natural pH of the vaginal flora (4 to 4.5).

In typical embodiments, the planar product for treatment of the urogenital tract has a shape which is configured such that it is placeable and wearable interlabially. From the shape of various sanitary pads for example, a person skilled in the art is aware of which shapes are advantageous in this connection. For instance, the planar product can have an elongated shape, especially with rounded corners, or be oval, round, especially circular, rectangular or square. Moreover, the planar product according to the invention can include so-called wings, as is known to a person skilled in the art.

The planar product can cover the urogenital space in the operative state, at least in the region of the vulva, of the vagina and of the urethra. Typically, the shape of the planar product can follow the realities of the anatomy in the urogenital space.

Preferably, the planar product has an elongated shape which is typically rounded at the ends.

In one embodiment of the invention, the planar product for treatment of the urogenital tract comprises multiple textile layers. They can respectively consist of different materials or else of the same materials in full or in part. The multiple layers are typically joined to one another by bonding, stamping, welding, sewing or with the aid of microwaves or ultrasonic waves. In advantageous embodiments, the distal face of the planar product is formed from a textile layer composed of a hydrophobized material or a further hydrophobic coat. If the planar product includes multiple textile layers, at least one layer can include an absorbent material, such as, for example, absorbent cotton or some other absorptive material. It can, for example, form the middle layer in a three-ply structure, or be preferably coated with a hydrophobic material in a two-ply structure.

It is moreover possible for a planar product according to the invention to be directly applied to a commercially available sanitary pad in a suitable shape, for example by bonding, sewing or stamping.

In a further aspect, the invention provides a planar product according to the invention as per the described embodiments for use in therapy or prophylaxis. More particularly, such a planar product can be used in the therapeutic or prophylactic treatment of infectious diseases of the urogenital area. What is particularly advantageous is a planar product according to the invention for use in the therapy or prophylaxis of bladder, kidney and urinary tract inflammations.

In a further aspect, the invention provides a method for producing a planar product according to the invention for treatment of the urogenital tract, including the steps of: (a) providing at least one textile layer; (b) coating a proximal face with an antimicrobially and/or antiseptically effective agent, such that the planar product has a vitality of >80% after 18 h of incubation time in the cytotoxicity test described here and an efficacy of >80% in the PI-MIA test described herein.

In a preferred embodiment, the method according to the invention additionally includes the step of (c) joining multiple textile layers by stamping, welding, bonding, sewing or with the aid of microwaves and ultrasonic waves.

In a further embodiment, the method according to the invention additionally includes the step of (d) applying bioadhesive and/or microadhesive agents on the proximal face.

A person skilled in the art will understand that the alphabetic listing of the steps does not reveal the order of the steps. For example, the proximal face can be coated first via step (b) and then via step (d), in reverse order, or else simultaneously.

Typically, the antimicrobially and/or antiseptically effective agent is provided by mixing the respective components, as described above, preferably in the disclosed concentrations in aqueous solution.

The one or more textile layers joined to one another can, for example, be coated at least on the proximal face with the antimicrobially and/or antiseptically effective agent by immersion, spreading, printing, spraying, dripping or by similar methods.

Ways of Carrying Out the Invention

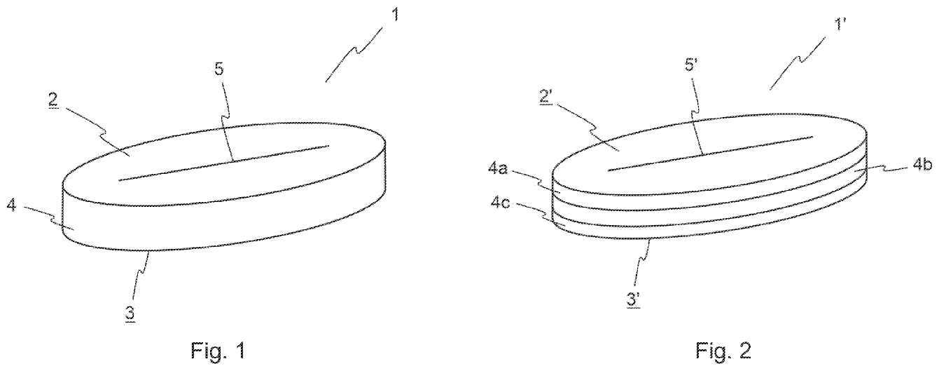

FIG. 1 shows a schematic view of a planar product according to the invention for treatment of the urogenital tract 1 having a proximal face 2 and a distal face 3. The planar product 1 includes a textile layer 4 (for a better overall view, this is depicted in a distinctly broadened manner). The proximal face 2 is coated with an antimicrobially and/or antiseptically effective agent (not depicted). In the exemplary embodiment shown, the proximal face additionally comprises a top stitching 5.

FIG. 2 depicts a further embodiment of a planar product for treatment of the urogenital tract 1′ having a proximal face 2′ and a distal face 3′. The planar product 1′ includes three textile layers 4a, 4b and 4c, which are, for example, joined to one another by sewing, bonding, stamping, etc. (for a better overall view, this is depicted in a distinctly broadened manner). Here, it is particularly advantageous when the middle layer 4b consists of an absorbent material, such as absorbent cotton or a foam material. The layer 4c in particular can be hydrophobized by a hydrophobic coating. Furthermore, the proximal face 2′ comprises a top stitching 5′ in the longitudinal direction.

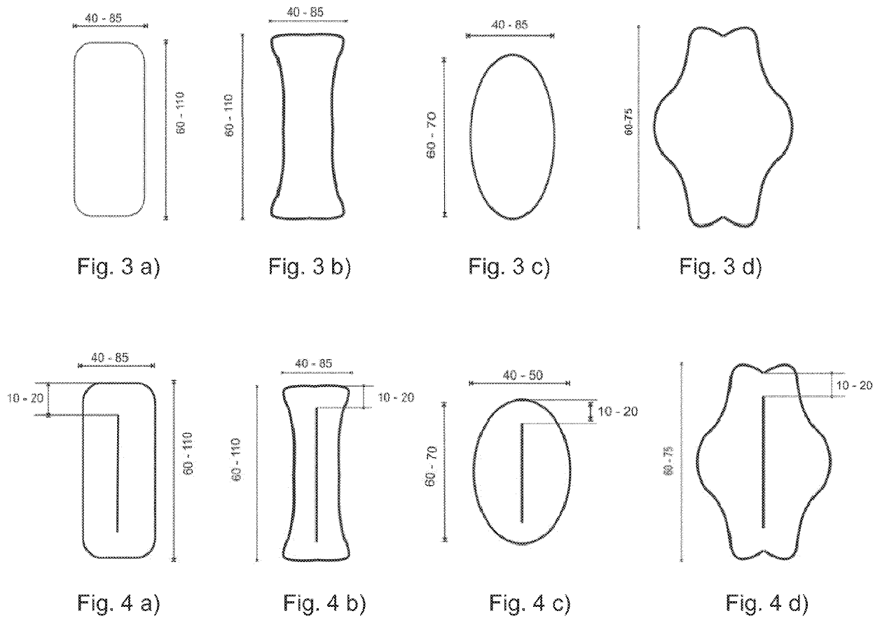

FIG. 3 shows a top view of various different embodiments of a planar product according to the invention for treatment of the urogenital tract. The planar product shown in FIG. 3 a) has an elongated shape which is rounded at the corners. In FIG. 3 b), the shape of the planar product according to the invention is likewise elongated, but the edges of the longitudinal sides are inwardly curved and hence of a concave shape in profile in the top view. Such an embodiment can, for example, ensure increased wear comfort, since freedom of movement, especially of the legs, is additionally increased. FIG. 3 c) shows a planar product according to the invention that has an oval shape. Such an embodiment offers the advantages of a relatively large area, the result being that the proportion of the antimicrobially and/or antiseptically effective agent can be increased. Furthermore, FIG. 3 d) shows one embodiment of the invention with wings, in which the two longitudinal sides each comprise one wing, as is known from common sanitary pads. Depending on the requirements, the in FIG. 3 a) to d) have a length of 60 to 110 mm and a width of 40 to 85 mm However, square or circular planar products are also provided, or planar products which cover a larger area and can accordingly have a length or a diameter of 60 up to 200 mm.

FIG. 4 shows in a top view of the respective proximal faces of some further embodiments of the invention. In this case, the proximal face comprises a top stitching in the longitudinal direction of the planar product.

Such a top stitching in the longitudinal direction, i.e., in the direction of the labia in the operative state, has the advantage that the contact area in relation to the skin is enlarged and the planar product fits tightly on the skin. Furthermore, bending is facilitated and slipping during wearing is more efficiently avoided. In the typical embodiments shown, the length of the top stitching is about ⅔ of the length or diameter of the planar product. For example, the distance of the top stitching from the edge of the planar product is, as depicted in FIGS. 4 a) to d), typically 10 to 20 mm, in the case of a length of the planar product of 60 to 110 mm

To determine the compatibility of a planar product for treatment of the urogenital tract, the Cytotoxicity Test described below can be used. It is based on the standard DIN ISO 10993-5.

Reconstituted human 3D EpiVaginal models (e.g., MatTek) are used. To this end, an element of 6 mm in diameter from a planar product according to the invention is topically applied to the 3D EpiVaginal skin models and incubated on the models for 3 and 18 hours. Skin models treated with sterile water are used as negative control, and skin models treated with the anionic surfactant Triton X-100 (1%) are used as positive control. The vitality of the skin models is evaluated in the MTT assay (3-(4,5-dimethylthiazol-2-yl)-2,5-diphenyltetrazolium bromide) (see Stockert et al. Acta Histochemica 120: 159-167 (2018), doi: 10.1016/j.acthis.2018.02.005; Chacon et al. In Vitro Methods in Pharmaceutical Research, ISBN 9780080534602 and https://en.wikipedia.org/wiki/MTT_assay). This involves setting the vitality value of the negative control to 100%. The positive control typically shows a vitality of 25% after 3 h of incubation and a vitality of less than 10% after 18 h. According to the invention, what is considered to be an acceptable value is typically a vitality of >80% after 18 h.

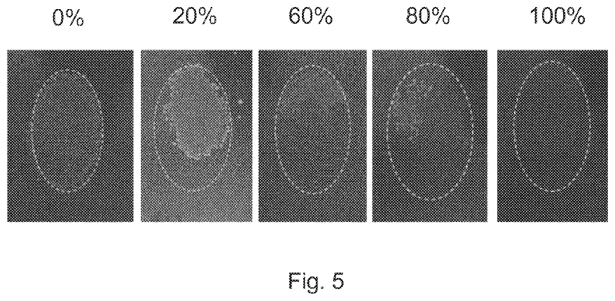

The efficacy of a planar product for treatment of the urogenital tract can be determined on the basis of the test for determination of pad-inherent microbial inhibition activity (PI-MIA Test) that is described below. In the PI-MIA test, a fresh overnight culture of E. coli (DSM 498) is first grown. Moreover, a test solution is prepared in sterile water with 105 CFU/mL (CFU=total pathogen count). Thereafter, 500 μl ut each of E. coli with 105 CFU/mL is applied to a sterile test surface. Meanwhile, a device for treatment of the urogenital tract is prepared, for example a planar product according to the invention that has been coated with 400 viL of an antimicrobially and/or antiseptically effective agent according to the invention, and also a 0 control (typically a device coated with water). The incubation than takes place at room temperature for 60 min in each case. Thereafter, the devices (sample and 0 control) are removed and contacted, with the bacterially contaminated contact side, onto an LB plate (lysogeny broth plate) and incubated at room temperature for 5 min. The devices are then removed and the LB plates are incubated at 37° C. for 18 hours. Lastly, the sample can be compared with the 0 control. The PI-MIA values are moreover determined via a reference scale. Lastly, the sample can be compared with the 0 control. The PI-MIA values are moreover determined via a reference scale as per FIG. 5.

A sufficient effect is typically provided at 80% growth inhibition.

The following table shows a comparative examples of the antimicrobially and/or antiseptically effective agent used:

| | Example 1 | Example 2 | Example 3 | Example 4 |
|---|---|---|---|---|
| g/l | [g/L] | [g/L] | [g/L] | [g/L] |
| Vitamin C | 0.8806 | 0.8806 | 0.8806 | 0.8806 |
| Tartaric acid | 5.2530 | 5.2530 | 3.0000 | 5.2530 |
| Citric acid | 6.7250 | 3.8400 | 3.8400 | 6.7250 |
| Trisodium citrate | | 4.4150 | | |
| Copper(II) chloride | 0.8524 | 0.8524 | 0.8524 | 0.8524 |
| ZnSO₄ | | | | |
| FeSO₄ | | | | |
| Sodium hydroxide | | | | 2-3 g/L |
| Sodium dodecyl | 2.5000 | 2.5000 | 2.5000 | 2.5000 |
| sulfate | | | | |
| pH | ~1.9 | ~2.9 | ~2.3 | ~2.9 |
| PI-MIA test | — | not 100% | ok | ok |
| Cytotoxicity/cell | 64.9 | 61.1 | 87.1 | 74.6 |
| vitality after 3 h | | | | |
| Cytotoxicity/cell | 54.2 | 41.7 | 81.7 | 67.5 |
| vitality after 18 h | | | | |

It becomes apparent that the use of tartaric acid in a concentration of not more than 6 g/L has generally an advantageous effect on vitality. Here, the replacement of CuCl with FeSO4 (concentration of 0.9632 g/L) and ZnSO₄ (concentration 1.0235 g/L) comparable values.

1. A planar product for treatment of the urogenital tract comprising a proximal face and a distal face wherein the planar product comprises at least one textile layer and wherein the proximal face is coated with at least one antimicrobially and/or antiseptically effective agent, and wherein the coated proximal face of the planar product has a vitality of >80% after 18 h of incubation time in a Cytoxicity Test according to DIN ISO 10993-5 and an efficacy of >80% in a Pad-Inherent Microbial Inhibition Activity PI-MIA Test.

2. The planar product as claimed in claim 1, wherein the proximal face comprises top stitching in the center in the longitudinal direction for facilitation of bending of the planar product.

3. The planar product as claimed in claim 1, wherein the proximal face is coated with bioadhesive and/or mucoadhesive agent(s) in stripes or in a punctiform manner for better fixation on skin.

4. The planar product as claimed in claim 1, wherein the at least one antimicrobially and/or antiseptically effective agent comprises at least one of surfactant(s), quaternary ammonium compound(s), biguanide(s), organic acid(s), metal ions, iodine compound(s), aldehyde(s) of mixtures thereof.

5. The planar product as claimed in claim 1, wherein the at least one antimicrobially and/or antiseptically effective agent comprises tartaric acid in a concentration of 1 to 5 g/L, or 2 to 4 g/L.

6. The planar product as claimed in claim 1, wherein the at least one antimicrobially and/or antiseptically effective agent comprises: at least one vitamin selected from vitamin A, B, C, E; at least one metal ion of group eight, eleven or twelve of the periodic table; at least one aliphatic or aromatic organic acid or derivatives thereof; at least one surface-active compound; optionally bases and/or buffer systems for pH adjustment.

7. The planar product as claimed in claim 1, wherein the at least one antimicrobially and/or antiseptically effective agent comprises at least one gel former.

8. The planar product as claimed in claim 7, wherein the at least one gel former comprises at least one of polysaccharide(s), silicon dioxide, polyvinylpyrolidone(s), polyvinyl alcohol(s), polymethacrylate(s), poly(N-isopropylacrylamides), polyacrylic acid(s) or mixtures thereof.

9. The planar product as claimed in claim 1, wherein the at least one antimicrobially and/or antiseptically effective agent comprises, ascorbic acid as vitamin, copper as metal ion, citric and/or tartaric acid as organic acid, lauryl sulfate(s) as surfactant and optionally agarose or carrageenan as gel former.

10. The planar product as claimed in claim 1, wherein the at least one antimicrobially and/or antiseptically effective agent comprises
0.5 to 1.5 g/L of at least one vitamin; and/or
2 to 6.5 g/L of at least one aliphatic or aromatic acid; and/or
0.5 to 1.5 g/L of metal ions; and/or
1.5 to 10 g/L of surfactant(s); and/or
2.5 to 45 g/L of at least one gel former.

11. The planar product as claimed in claim 1, wherein the at least one antimicrobially and/or antiseptically effective agent has a pH between 2.3 to 3.1.

12. The planar product as claimed in claim 1, wherein the planar product has a shape which is configured such that the planar product is placeable or wearable interlabially.

13. A product for treatment of the urogenital tract comprising the planar product as claimed in claim 1.

14. A method for producing a planar product for treatment of the urogenital tract, comprising:
(a) providing at least one textile layer;
(b) coating a proximal face of the planar product with at least one antimicrobially and/or antiseptically effective agent, such that the planar product has a vitality of >80% after 18 h of incubation time in a Cytotoxicity Test according to DIN ISO 10993-5 and an efficacy of >80% in a PI-MIA Test.

15. The method as claimed in claim 14, further comprising:
(c) joining multiple textile layers by stamping, welding, bonding, sewing or with the aid of microwaves or ultrasonic waves.

16. The method as claimed in claim 14, further comprising:
(d) applying bioadhesive and/or mucoadhesive agents on the proximal face.

17. The planar product as claimed in claim 1, wherein the metal ions comprise copper, zinc, iron, silver, ruthenium, and/or rhodium.

18. The planar product as claimed in claim 8, wherein the at least one polysaccharide(s) comprises agarose, carrageenan, pectin, xanthan gum, alginic acid and/or alginate(s).
